(19) United States
(12) Plant Patent Application Publication
(10) Pub. No.: US 2008/0244796 P1
(43) Pub. Date: Oct. 2, 2008

(54) Male asparagus hybrid 'M256'

(75) Inventors: Mikeal Roose, Riverside, CA (US); Neil K. Stone, Temecula, CA (US); Laverne F. Lippert, Anacortes, WA (US); Janice M. Gish, Anacortes, WA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: The Regents of the University of California Office of the President, Oakland, CA (US)

(21) Appl. No.: 11/732,506

(22) Filed: Apr. 2, 2007

(51) Int. Cl. A01H 5/00

(52) U.S. Cl. PLT/260

(57) ABSTRACT

This invention relates to a new and distinctive male asparagus hybrid called ‘M256’. ‘M256’ has the ability to confer desirable traits on its progeny, such as early spear emergence, and produces offspring that have a higher proportion of marketable spears.

### LATIN NAME OF THE GENUS AND SPECIES

Asparagus officinalis.

### VARIETY DENOMINATION

The variety denomination is ‘M256’.

### BACKGROUND OF THE INVENTION

Asparagus is a dioecious species with individual plants being either male or female. Asparagus cultivars that have been most commonly used for fresh market green asparagus in the major growing regions of California include, Atlas, Grande, Ida Lea, and UC157. These cultivars are all produced from crossing a genetically unique male clone with a genetically unique female clone to produce F1 seed.

### BRIEF SUMMARY OF THE INVENTION

‘M256’ is a new and distinct male asparagus hybrid that is used to produce asparagus cultivars that have commercially desirable traits. ‘M256’ can be distinguished from the closest varieties known to the inventor by its ability to produce offspring that have a higher proportion of marketable spears with tightly appressed bracts, and a significantly higher total yield when yields are added over several years. The variety is further distinguished by its ability confer early spear emergence to its progeny.

### BRIEF DESCRIPTION OF THE DRAWINGS

FIG. 1 provides an illustration of a typical stalk from an asparagus plant. The Figure illustrates the relative positions on the plant and the source of the data presented below. References are made with the use of brackets and letters, the same letters which are also listed in parentheses under the stalk data.

FIG. 2 shows typical foliage of ‘M256’. The photograph depicts a 10 year-old plant of ‘M256’ growing in Riverside, Calif.

### DETAILED DESCRIPTION OF THE INVENTION

Pedigree ‘M256’ was selected as a single plant in 1982 from the hybrid ‘Ida Lea’, which is from a cross between asparagus cultivars ‘F189’ and ‘M138’. ‘F189’ is from a cross between ‘F111’ and M.O.P. (Male Open Pollinated). ‘F111’ a single plant selection from ‘UC66’. ‘M138’ is from a cross between ‘F120’ and M.O.P. ‘F120’ is a single plant selection from ‘UC309’.

‘M256’ was selected as a single plant and propagated by tissue culture. ‘M256’ was used in a hand cross with ‘F189’ in 1982. In 1987 ‘M256’ was planted into a female block in Field 6AS. ‘M256’ conferred early spear emergence to its progeny. In 1989 ‘M256’ asparagus crowns were transferred to a female holding block sufficiently isolated from other asparagus on the field station to produce hybrid seed of the genetic purity required for use in a field trial to evaluate ‘M256’ offspring. In 1990 the seed resulting from pollination of many of the females in the holding block with ‘M256’ was used to plant seedlings in a new trial.

Cultivation ‘M256’ can be clonally propagated by crown division. The distinguishing characteristics of ‘M256’ are stable and reproduce true to type in successive generations. ‘M256’ is usually grown in isolated plantings of clonally propagated plants along with a female clone to produce F1 seeds. Recommended cultural practices for ‘M256’ are similar to those of other asparagus plantings for seed production. Plantings can be established using 8-week old seedling transplants or 1-year-old crowns. Performance can be good on soils with low (Riverside, Calif.) or high (Delta regions of California) organic content. Rust and asparagus aphid should be controlled with spray regimes. Periodic genetic tests of parent clones are typically conducted to verify genetic purity, or identify any problems that may arise. Some ways of maintaining genetic purity include hand harvesting of stalks with mature seed, planting clones in rows of all males and all females so as to be able to identify any volunteers of the opposite sex within the row, practicing no-till farming, applying pre-emergent chemicals to inhibit seed germination, periodic inspections and seedling removal by hand.

Plant Characteristics

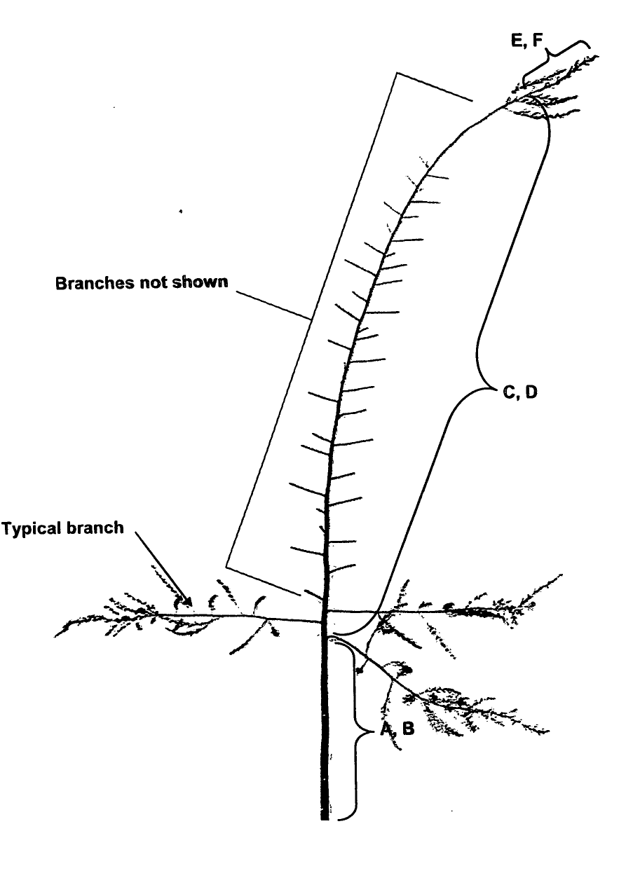

The attached photograph, FIG. 1, illustrates the relative positions on the plant and the source of the data presented below. References are made with the use of brackets and letters, the same letters which are also listed in parentheses under the stalk data.

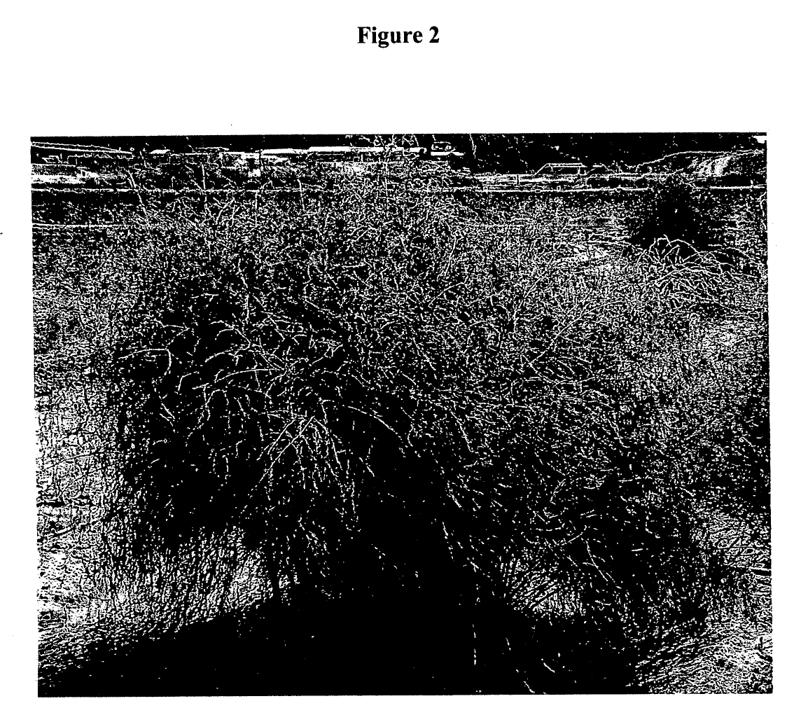

Typical foliage of M256 is shown in FIG. 2.

The following data were obtained from the longest stalk and are the mean value of measurements on five separate plants unless otherwise indicated.

The colors presented below refer to colors in The Royal Horticultural Society color chart and are the most common colors observed among the plants measured.
Stalk data:
   Number of nodes below the first branch (A).—27.6.
   Number of cm from crown to the first branch (B).—57.2.
   Number of branches (C).—55.8.
   Number of cm between first and last branch (D).—116.4.
   Internode length in cm between branches (D/(C−1)).—2.132.
   Number of cladophyll nodes beyond the last branch (E).—47.6.
   Number of cm beyond the last branch (F).—24.1.
   Internode length in cm beyond the last branch (F/(E−1)).—0.52.
   Largest stalk diameter in mm.—16.42.
   Mean diameter of three largest stalks in mm.—14.72.
   Number of stalks.—60.6.
   Stalk vigor index (stalk number)(mean diameter)².—13402.71.
   Mature stalk color, bloom removed.—144A.
Flower data:
   Petal tip color (yellow).—yellow 1C.
   Petal base color (green).—yellow-green 146C.
   Flower length in mm.—5.7.
   Flower width at midpoint in mm.—2.58.
Cladophyll data:
   Number per node.—3.3.
   Length in mm.—13.96.
   Width in mm.—0.27.

What is claimed is:

1. A new and distinct male asparagus hybrid having the characteristics essentially as described and illustrated herein.
